(19) United States
(12) Patent Application Publication
(10) Pub. No.: US 2019/0365326 A1
(43) Pub. Date: Dec. 5, 2019

(54) TRANSVASCULAR PRESSURE SENSING DEVICE

(71) Applicant: RTM Vital Signs LLC, Fort Washington, PA (US)

(72) Inventor: Josiah VERKAIK, Boise, ID (US)

(21) Appl. No.: 16/423,515

(22) Filed: May 28, 2019

Related U.S. Application Data
(60) Provisional application No. 62/678,296, filed on May 31, 2018.

(51) Int. Cl. A61B 5/00; A61B 5/0235; A61B 5/0215

(52) U.S. Cl. CPC A61B 5/6876; A61B 5/02241; A61B 5/0215; A61B 5/0235

(57) ABSTRACT

A transvascular pressure sensing device including a housing having a proximal portion and a distal portion opposite the distal portion, the proximal portion including a blood pressure sensor disposed therein, a compliant balloon surrounding at least a portion of the housing and defining a fluid chamber therebetween, the fluid chamber including a first portion defining a first volume and a second portion defining a second volume greater than the first volume, and a fluid disposed within the fluid chamber and exerting a hydraulic pressure on the balloon, the blood pressure sensor being configured to measure a change in the hydraulic pressure when a force is distributed over the second portion of the balloon.

### CROSS-REFERENCE TO RELATED APPLICATION

This application is related to and claims priority to U.S. Provisional Patent Application Ser. No. 62/678,296, filed May 31, 2018, entitled TRANSVASCULAR PRESSURE SENSING DEVICE, the entirety of which is incorporated herein by reference.

### STATEMENT REGARDING FEDERALLY SPONSORED RESEARCH OR DEVELOPMENT

n/a

### FIELD

The present disclosure relates to a device and method for communicating a hydraulic blood pressure from within a blood vessel to outside of the blood vessel.

### BACKGROUND

A patient's vital signs provide information useful for detecting and/or monitoring medical conditions. Known devices for measuring vital signs include, for example, blood pressure cuffs which attach to a patient's upper arm, pulse oximeters designed to be placed around the patient's fingertip, thermometers for measuring body temperature, stethoscopes for observing biological sounds, tonometers, and pressure transducers including those in the form of transvascular devices having metal diaphragms.

Known pressure transducers typically include a housing containing a pressure measuring system, a diaphragm, and a substance, such as oil, for transferring a pressure exerted on the diaphragm to the pressure measuring system. The manufacturing of such pressure transducers may be difficult as a deflection of the diaphragm at the diaphragm's center should be no greater than a thickness of the diaphragm for accuracy. Further, metal diaphragms are known to present problems, such as lack of sensitivity in addition to manufacturing limitations. For example, metal diaphragms having a diameter of approximately 1.0 millimeter are too small to compensate for relatively small variations in deflection leading to non-compliance. Moreover, known transvascular devices typically necessitate the presence of significant hardware inside the blood vessel that may be hazardous for the patient including blood flow obstructions and the potential for thrombus formation.

### SUMMARY

The present invention advantageously provides a transvascular pressure sensing device having a housing including a proximal portion and an opposing distal portion, the proximal portion including a blood pressure sensor disposed therein, a compliant balloon surrounding at least a portion of the housing and defining a fluid chamber between the balloon and the portion of the housing, the fluid chamber including a first portion defining a first volume and a second portion defining a second volume greater than the first volume, and a fluid disposed within the fluid chamber and exerting a hydraulic pressure on the balloon, the blood pressure sensor being configured to measure a change in the hydraulic pressure when a force is distributed over the second portion of the balloon.

In another aspect of the invention, the balloon is made of a flexible polyurethane material having a width between 90 to 110 micrometers.

In another aspect of the invention, the balloon is configured to provide a uniform hydraulic pressure between the first portion and the second portion of the fluid chamber.

In another aspect of the invention, the housing is elongated and of a cylindrical shape.

In another aspect of the invention, the proximal portion and the distal portion of the housing define a housing axis extending therethrough, and the second portion of the fluid chamber is transverse with respect to the housing axis.

In another aspect of the invention, the balloon defines a partially enclosed internal channel extending from the proximal portion to the distal portion, and the distal portion defines a plurality of apertures extending therearound.

In another aspect of the invention, the proximal portion of the housing includes a sensor casing in fluid communication with the balloon and having the blood pressure sensor disposed therein.

In another aspect of the invention, the blood pressure sensor is a pressure sensitive MEMS device.

In another aspect of the invention, the first portion of the fluid chamber contours the proximal portion of the housing and the second portion of the fluid chamber extends away from the distal portion of the housing.

In another aspect of the invention, the second portion of the fluid chamber narrows in a direction away from the housing.

In another aspect of the invention, the fluid chamber is of an L-shape.

In another configuration, the present invention includes a blood pressure communication device having a blood pressure probe including a housing having a proximal portion, a distal portion opposite and proximal portion, and defining an internal channel extending therebetween, a balloon enclosing the internal channel, the balloon defining a tapered fluid chamber extending away from the distal portion of the housing, a fluid stored within the balloon, the fluid characterized by a hydraulic pressure equal to a pressure applied to the balloon, and a pressure sensor in communication with the housing, the pressure sensor being configured to measure the hydraulic pressure.

In another aspect of the invention, the balloon is configured to be disposed within a blood vessel and the pressure sensor is configured to be disposed outside of the blood vessel.

In another aspect of the invention, the fluid is a silicone.

In another aspect of the invention, the balloon is made of a polyurethane material.

In another aspect of the invention, the pressure sensor is a pressure sensitive MEMS device.

In another aspect of the invention, the distal portion of the housing defines an aperture extending therethrough, the aperture being in fluid communication with the internal channel.

In another aspect of the invention, the balloon is of an L-shape tapering in a direction away from the housing.

In another aspect of the invention, the balloon defines a circumference between 0.5 to 1.5 millimeters at the proximal portion of the housing.

In an additional configuration, the present invention includes an implantable blood pressure sensor having an elongated cylindrical housing including a first distal portion having a tip and a proximal portion opposite the distal portion, a flexible diaphragm at least partially surrounding the housing and including an extension member extending away from the housing, the extension member defining a fluid chamber between the flexible diaphragm and the second portion of the housing, a non-compressible fluid disposed within the fluid chamber, the non-compressible fluid including a hydraulic pressure equal to a pressure applied to the diaphragm, and a pressure sensor disposed within the tip of the housing for measuring a change in the hydraulic pressure resulting from the pressure applied to the diaphragm.

### BRIEF DESCRIPTION OF THE DRAWINGS

A more complete understanding of the present application, and the attendant advantages and features thereof, will be more readily understood by reference to the following detailed description when considered in conjunction with the accompanying drawings wherein:

FIG. 1 is a side view of a blood pressure communication device including a blood pressure probe having a housing and a compliant balloon surrounding at least a portion of the housing in accordance with the present invention;

FIG. 2 is a side cross-sectional view of the blood pressure communication device taken along section A-A of FIG. 1 and being disposed within an exemplary blood vessel in accordance with the present invention;

FIG. 3 is a perspective bottom view of the blood pressure communication device of FIG. 1 in accordance with the present invention;

FIG. 4 is a partial perspective bottom view of the blood pressure communication device of FIG. 1 disposed within an exemplary blood vessel in accordance with the present invention; and

FIG. 5 is a partial side view of the blood pressure communication device of FIG. 1 disposed within an exemplary blood vessel in accordance with the present invention.

### DETAILED DESCRIPTION

Before describing in detail exemplary embodiments, it is noted that the embodiments reside primarily in combinations of device and method steps related to communicating a hydraulic blood pressure from within a blood vessel to outside of the blood vessel. Accordingly, the device and method steps have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

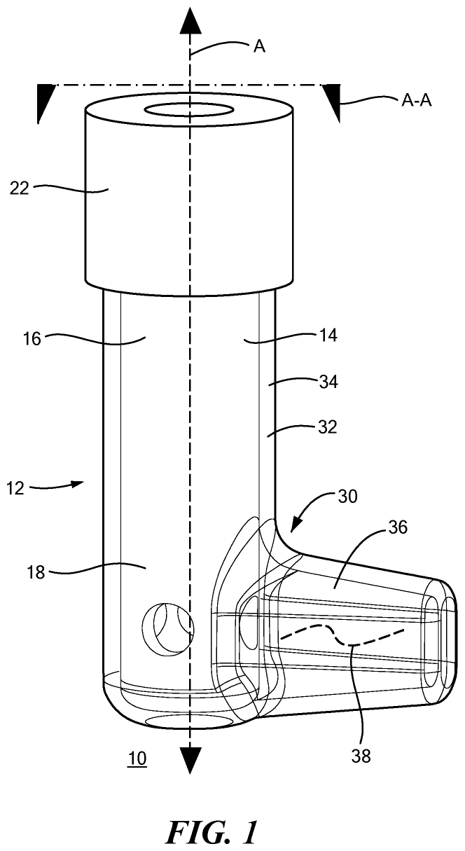

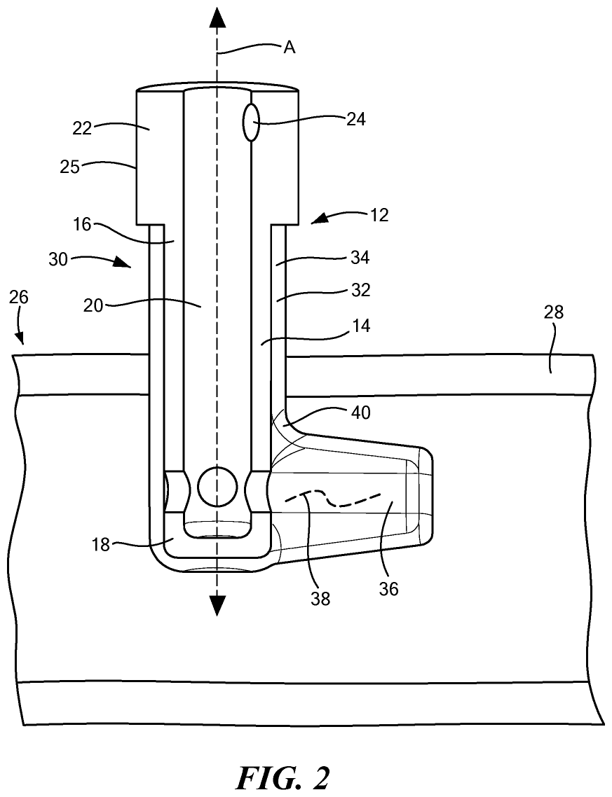

Referring now to the drawings in which like reference designators refer to like elements there is shown in FIGS. 1-2 an exemplary blood pressure communication device generally designated as “10.” FIG. 1 depicts a side view of the device 10 and FIG. 2 depicts a side cross-sectional view of the device 10 taken along section A-A of FIG. 1. The blood pressure communication device 10 may be referred to herein as a transvascular pressure sensing device or “the device 10” and includes a blood pressure probe 12 configured to be at least partially implanted in a blood vessel of a patient, such as a human or animal patient, for communicating a hydraulic pressure within the blood vessel to a pressure sensor outside of the blood vessel.

The probe 12 may include a housing 14 having a proximal portion 16 and a distal portion 18 opposite the proximal portion 16. The proximal portion 16 and the distal portion 18 define a housing axis “A” extending therethrough and, as shown in FIG. 2, an internal channel 20 extending along the housing axis. The housing 14 is made of a biocompatible material, such as and without limitation, a polyurethane material that may be elongated and of a cylindrical shape, although other shapes are within the scope of the present invention.

The proximal portion 16 of the housing 14 may include a tip 22 or stem made of a rigid polyurethane material for housing a blood pressure sensor 24 therein. The blood pressure sensor 24 may be disposed within a sensor casing 25 in the proximal portion 16 of the housing. The sensor casing 25 may be made from a biocompatible material or other material that is safe to use inside the body for long or short periods of time. As depicted using an exemplary blood vessel 26 of FIG. 2, the blood pressure sensor 24 may be disposed outside of a wall 28 of the blood vessel 26, whereas the distal portion 18 may be transvascular, i.e., configured to be disposed through the wall 28 of the blood vessel 26. Although the pressure sensor 24 is depicted within the tip 22, in other configurations, the pressure sensor 24 may be disposed in a separate housing (not shown) in communication with the probe 12 or the pressure sensor may be disposed in another location in the device 10. In one configuration, the pressure sensor 24 is a pressure sensitive MEMS device, however other types of pressure sensors 24 may be used which are configured to measure hydraulic pressure. The term “hydraulic pressure” generally refers to fluid in a confined space, i.e. a closed system, wherein the fluid is a medium to transmit force. This is in accordance with the discovery of Pascal that a pressure applied to any part of a confined fluid transmits to every other part with no loss. The pressure acts with equal force on all equal areas of the confining walls in a direction perpendicular to the wall surfaces. The pressure sensor 24 may be coupled to one or more electronic circuits, a display, and the like (not shown) associated with power and operation of the pressure sensor 24.

A compliant balloon 30 surrounds at least a portion of the housing 14 and defines a fluid chamber 32 between the housing 14 and the balloon 30. In one configuration, the balloon 30 may be a flexible diaphragm that at least partially surrounds the housing 14. In other words, the housing 14 and the balloon 30 define a space or gap therebetween and the fluid chamber 32 is within the space or gap. In one configuration, the balloon 30 is made of a material such as a flexible polyurethane manufactured and sold under the name Biospan® having a width between 90 to 110 micrometers (“um”). In other configurations the balloon 30 may be made of another flexible material having various widths or thicknesses and may also be characterized as a flexible diaphragm. The sensor casing 25 may be in fluid communication with the balloon 30 and the blood pressure sensor 24 may be disposed within the sensor casing 25.

The fluid chamber 32 includes a first portion 34 surrounding some or all of the proximal portion 16 of the housing 14 and a second portion 36 surrounding the distal portion 18 of the housing 14 to at least partially enclose the internal channel 20. In particular, the first portion 34 of the fluid chamber 32 contours the housing 14, whereas the second portion 36 of the fluid chamber 32 extends away from the housing 14. The second portion 36 of the fluid chamber 32 may also be an extension member of the housing 14 that extends away from the housing 14 and the extension member may define a fluid chamber 32 between the balloon 30/flexible diaphragm and the distal portion 18 of the housing 14. For example, the first portion 34 of the fluid chamber 32 contours the proximal portion 16 of the housing and the second portion 36 of the fluid chamber 32 extends away from the distal portion 18 of the housing 14. As such, the first portion 34 defines a first volume and the second portion 36 defines a second volume greater than the first volume for storing a fluid 38 that exerts or exhibits a hydraulic pressure on the balloon 30. The fluid 38 may be a non-compressible fluid such as, without limitation, a silicone.

When the distal portion 18 of the probe 12 and the second portion 36 of the fluid chamber 32 are disposed within the blood vessel 26, the hydraulic pressure of the fluid 38 within the fluid chamber 32 is approximately equal to a force or pressure applied to the balloon 30 by the blood vessel 26. Such hydraulic pressure, including any changes to the hydraulic pressure as a result of the force from the blood vessel 26, may be measured by the blood pressure sensor 24 disposed outside of the blood vessel 26. In other words, the device 10 is configured to provide communication of the hydraulic pressure within the blood vessel 26 to the pressure sensor 24 outside of the blood vessel 26. The material of the balloon 30 is sufficiently thin and flexible to provide a uniform hydraulic pressure between the first portion 34 and the second portion 36 of the fluid chamber 32. In other words, the material and flexibility of the balloon 30 is sufficient to prevent a pressure differential between the fluid pressure within the balloon 30 and the blood pressure around the second portion 36 of the fluid chamber 32. Additionally, any fluid stored within the balloon 30 may have a hydraulic pressure of the fluid 38 that is equal to the pressure which is applied to the balloon 30. In one configuration, the non-compressible fluid may be characterized by a hydraulic pressure of the fluid 38 that is equal to the pressure applied to the balloon 30/flexible diaphragm.

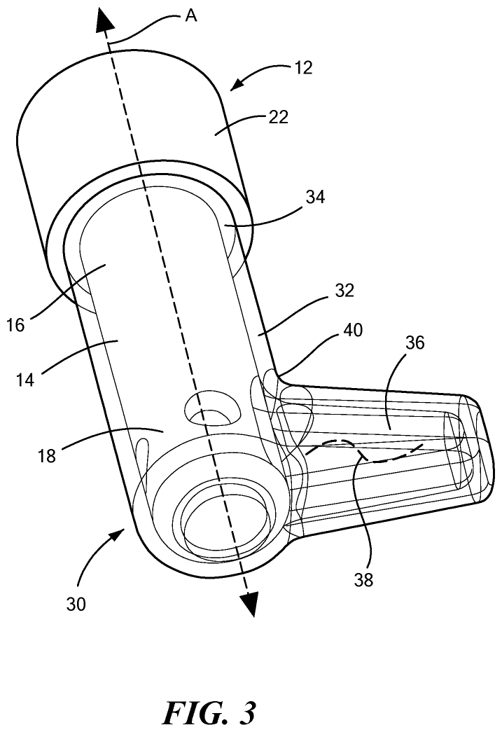

With reference to FIGS. 1-3, the second portion 36 of the fluid chamber 32 is depicted transverse with respect to the housing axis and including an L-shape that narrows or tapers in a direction away from the distal portion 18 of the housing 14. The structure of the balloon 30 allows the second portion 36 of the fluid chamber 32 to be isolated from the blood vessel wall 28 while being positioned in a blood flow path through the blood vessel 26. When the balloon 30 is covering the fluid chamber 32, the balloon 30 may have an L-shape that narrows or tapers in a direction away from the housing 14.

In one exemplary configuration, the balloon 30 defines a circumference between 0.5 to 1.5 millimeters around the housing 14 at a location adjacent a curved transition region 40 where the balloon expands to define the second portion 36 of the fluid chamber 32 having the L-shaped volumetric boundary with the volume greater than that of the first portion 34. The pressure sensitivity of the device 10 can be maintained despite small changes in mechanical properties of the polymer material of the device 10 and despite small changes in the volume of the fluid 38 as a result of the relatively large diaphragmatic surface of the second portion 36 of the fluid chamber 32 relative to the first portion 34.

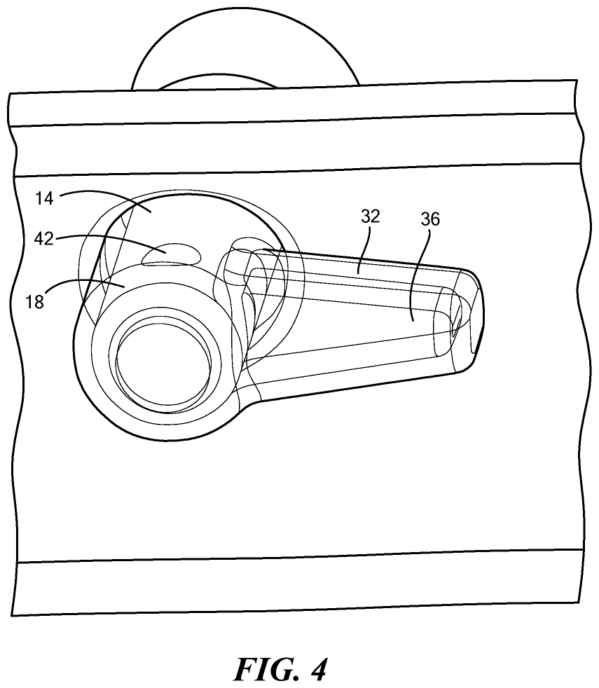

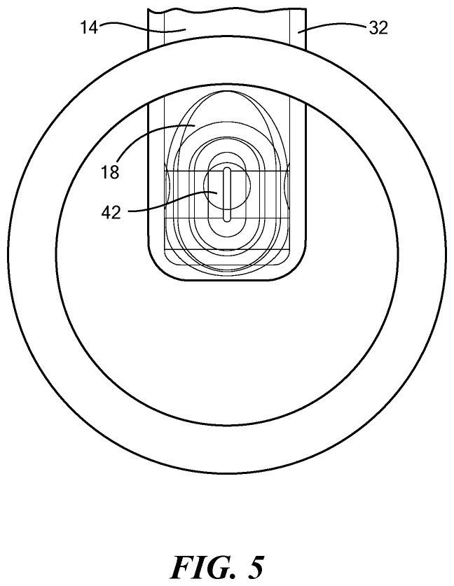

Referring now to FIGS. 4 and 5, the distal portion 18 of the housing 14 defines one or more apertures 42 extending therethrough, the apertures 42 being in fluid communication with the internal channel 20 (FIG. 2) to establish fluid communication between the second portion 36 of the balloon 30 and the pressure sensor 24 (FIG. 2). The internal channel 20 may be a partially enclosed and/or fully enclosed internal channel 20. The number of apertures 42 is not limited to a particular number and may include one aperture, 3-4 apertures extending around the circumference of the housing 14, or another configuration suitable for providing fluid communication along the internal channel 20. The balloon 30 may enclose the internal channel 20 and the balloon 30 may define a tapered fluid chamber 32 which extends away from the distal portion 18 of the housing 14.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A transvascular pressure sensing device comprising:
a housing including a proximal portion and a distal portion opposite the distal portion, the proximal portion including a blood pressure sensor disposed therein;
a compliant balloon surrounding at least a portion of the housing and defining a fluid chamber between the balloon and the portion of the housing, the fluid chamber including a first portion defining a first volume and a second portion defining a second volume greater than the first volume; and
a fluid disposed within the fluid chamber and exerting a hydraulic pressure on the balloon, the blood pressure sensor being configured to measure a change in the hydraulic pressure when a force is distributed over the second portion of the balloon.

2. The pressure sensing device of claim 1, wherein the balloon is made of a flexible polyurethane material having a width between 90 to 110 micrometers.

3. The pressure sensing device of claim 1, wherein the balloon is configured to provide a uniform hydraulic pressure between the first portion and the second portion of the fluid chamber.

4. The pressure sensing device of claim 1, wherein the housing is elongated and of a cylindrical shape.

5. The pressure sensing device of claim 1, wherein the proximal portion and the distal portion of the housing define a housing axis extending therethrough, and the second portion of the fluid chamber is transverse with respect to the housing axis.

6. The pressure sensing device of claim 1, wherein the balloon defines a partially enclosed internal channel extending from the proximal portion to the distal portion, and the distal portion defines a plurality of apertures extending therearound.

7. The pressure sensing device of claim 1, wherein the proximal portion of the housing includes a sensor casing in fluid communication with the balloon and having the blood pressure sensor disposed therein.

8. The pressure sensing device of claim 1, wherein the blood pressure sensor is a pressure sensitive MEMS device.

9. The pressure sensing device of claim 1, wherein the first portion of the fluid chamber contours the proximal portion of the housing and the second portion of the fluid chamber extends away from the distal portion of the housing.

10. The pressure sensing device of claim 9, wherein the second portion of the fluid chamber narrows in a direction away from the housing.

11. The pressure sensing device of claim 1, wherein the fluid chamber is of an L-shape.

12. A blood pressure communication device comprising:
a blood pressure probe including a housing having a proximal portion, a distal portion opposite the proximal portion, and defining an internal channel extending therebetween;
a balloon enclosing the internal channel, the balloon defining a tapered fluid chamber extending away from the distal portion of the housing;
a fluid stored within the balloon, the fluid characterized by a hydraulic pressure equal to a pressure applied to the balloon; and
a pressure sensor in communication with the housing, the pressure sensor being configured to measure the hydraulic pressure.

13. The blood pressure communication device of claim 12, wherein the balloon is configured to be disposed within a blood vessel and the pressure sensor is configured to be disposed outside of the blood vessel.

14. The blood pressure communication device of claim 12, wherein the fluid is a silicone.

15. The blood pressure communication device of claim 12, wherein the balloon is made of a polyurethane material.

16. The blood pressure communication device of claim 12, wherein the pressure sensor is a pressure sensitive MEMS device.

17. The blood pressure communication device of claim 12, wherein the distal portion of the housing defines an aperture extending therethrough, the aperture being in fluid communication with the internal channel.

18. The blood pressure communication device of claim 12, wherein the balloon is of an L-shape tapering in a direction away from the housing.

19. The blood pressure communication device of claim 12, wherein the balloon defines a circumference between 0.5 to 1.5 millimeters at the proximal portion of the housing.

20. An implantable blood pressure sensor comprising:
an elongated cylindrical housing including a proximal portion having a tip and a distal portion opposite the proximal portion;
a flexible diaphragm at least partially surrounding the housing and including an extension member extending away from the housing, the extension member defining a fluid chamber between the flexible diaphragm and the distal portion of the housing;
a non-compressible fluid disposed within the fluid chamber, the non-compressible fluid characterized by a hydraulic pressure equal to a pressure applied to the diaphragm; and
a pressure sensor disposed within the tip of the housing for measuring a change in the hydraulic pressure resulting from the pressure applied to the diaphragm.
